(19) United States
(12) Patent Application Publication
(10) Pub. No.: US 2007/0156200 A1
(43) Pub. Date: Jul. 5, 2007

(54) System and method for regulating blood pressure and electrolyte balance

(76) Inventors: Lilian Kornet, Maastricht (NL); Pierre A. Grandjean, Warsage (BE)

Correspondence Address: MEDTRONIC, INC., 710 MEDTRONIC PARKWAY NE, MINNEAPOLIS, MN, 55432-9924, US

(21) Appl. No.: 11/321,947

(22) Filed: Dec. 29, 2005

(51) Int. Cl. A61N 1/00

(52) U.S. Cl. 607/44

(57) ABSTRACT

The present invention is an apparatus and method for controlling blood pressure by stimulating the cardiac afferent sympathetic nerves. The invention may be implemented in a medical device having a pressure sensor for sensing blood pressure, an electrode for providing electrical signals to the cardiac afferent sympathetic nerves, and a controller for providing signals to the electrode as a function of blood pressure signals received from the pressure sensor.

### BACKGROUND OF THE INVENTION

The present invention relates generally to the field of medical devices, and more particularly to a medical device for regulating blood pressure and electrolyte balance through electrical stimulation of cardiac sympathetic afferent nerves.

Blood pressure naturally varies throughout the day, with minor variations being unremarkable. Large variances, however, are particularly troublesome. For example, persistent high blood pressure (“hypertension”) is a significant risk factor for heart failure, kidney disease, and renal failure, and abnormally low blood pressure (“hypotension”) is commonly associated with dizziness, seizures, and loss of consciousness.

The natural way for the body to respond to changes in blood pressure is through the baroreceptor reflex system, which receives afferent signals, or feedback, from baroreceptors, or sensors, located in the arteries of the upper body to detect changes in blood pressure and subsequently transmits efferent signals to the heart to cause a return to the body's desired baseline blood pressure. For example, when a person stands up quickly, he or she often experiences a decrease in blood pressure in the upper body. The baroreceptor reflex system responds by increasing the rate and force of the heart's contractions (thus increasing cardiac output) and by constricting blood vessels, thereby increasing blood pressure. Alternatively, to lower blood pressure, the baroreceptor reflex system decreases cardiac output and expands blood vessels.

The baroreceptor reflex system also activates sympathetic nerves to the kidneys. The renal body fluid feedback system helps to regulate blood pressure by causing the kidneys to excrete or retain water and electrolytes, particularly sodium. For example, an increase in blood pressure leads to increased excretion of sodium and water through the urinary system, with consequent reduction in blood volume, until blood pressure is returned to normal. A decrease in blood pressure leads to the kidneys conserving water and sodium until normal blood pressure is reached.

For many individuals, the body's own baroreceptor reflex system is sufficient to maintain blood pressure within acceptable limits. In many other individuals, however, the baroreceptor reflex system fails to adequately maintain safe blood pressures. Thus, a need exists to help the body control its blood pressure.

### BRIEF SUMMARY OF THE INVENTION

The present invention includes an apparatus and method for controlling blood pressure by stimulating the cardiac afferent sympathetic nerves. The invention may be implemented in a medical device having a pressure sensor for sensing blood pressure, an electrode for providing electrical signals to the cardiac afferent sympathetic nerves, and a controller for providing signals to the electrode as a function of blood pressure signals received from the pressure sensor.

### BRIEF DESCRIPTION OF THE DRAWINGS

FIG. 1 is a diagrammatic view of a patient with an external medical device for controlling the blood pressure and electrolyte balance of the patient in accordance with the present invention.

FIG. 2 is a diagrammatic view of a patient with an implantable medical device implanted therein for controlling the blood pressure and electrolyte balance of the patient in accordance with the present invention.

FIG. 3 is a simplified block diagram of a controller for the medical devices illustrated in FIGS. 1 and 2.

FIG. 4 is a flow diagram of a control routine that may be performed by the controller of FIG. 3 for controlling blood pressure and electrolyte balance.

### DETAILED DESCRIPTION

As described above, blood pressure is naturally regulated by the body's baroreceptor reflex system, which receives afferent signals from baroreceptors located in the arteries of the upper body to detect changes in blood pressure and subsequently transmits efferent signals to the heart to cause a return to the body's desired baseline blood pressure. These baroreceptors are innervated with sympathetic afferent nerves coming from the heart, stimulation of which reduces arterial baroreflex control of both the heart and the kidney.

The present invention is an apparatus and method for controlling blood pressure by stimulating the sympathetic afferent nerves innervating the baroreceptors. The invention may be implemented in a medical device having a pressure sensor for sensing blood pressure, an electrode for providing electrical stimulation to the cardiac afferent sympathetic nerves, and a controller for providing signals to the electrode as a function of blood pressure signals received from the pressure sensor.

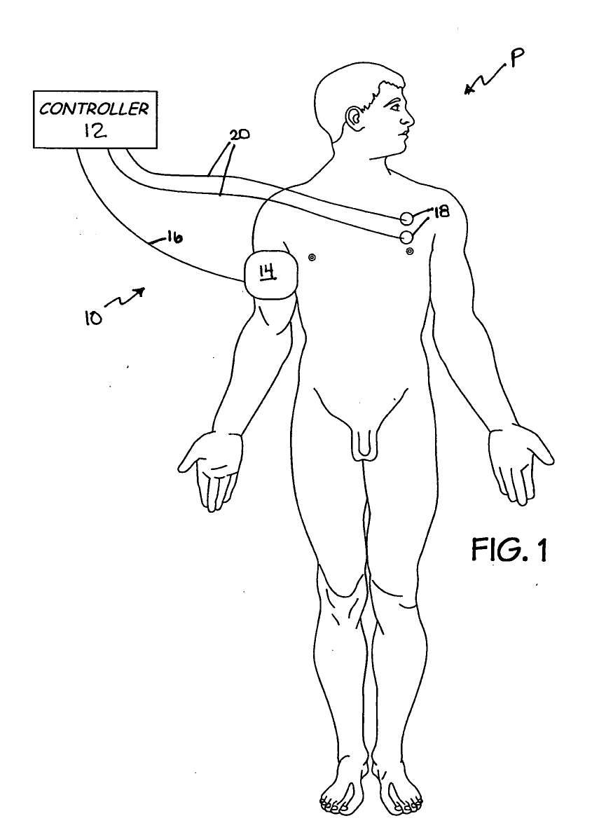

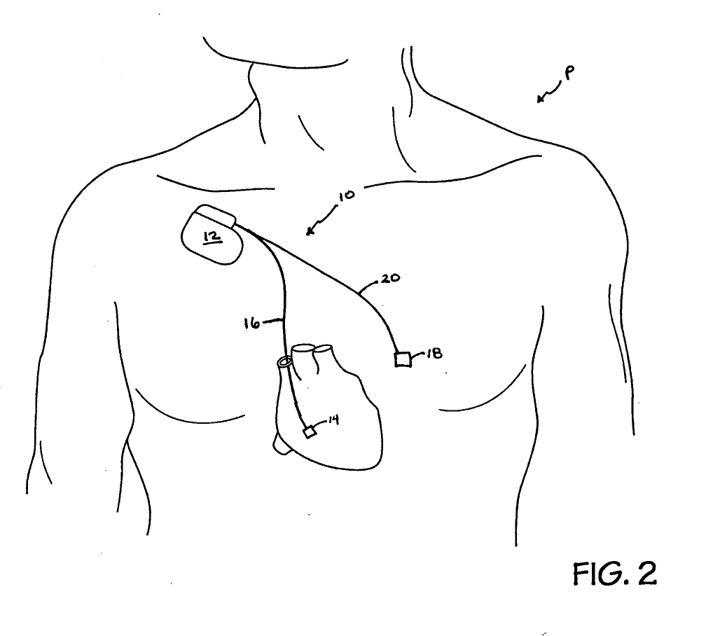

FIGS. 1 and 2 are diagrammatic views of patient P with medical device 10 for controlling current blood pressure and electrolyte balance of patient P in accordance with the present invention. In particular, FIG. 1 is an anterior diagrammatic view of patient P with medical device 10 as an external medical device (XMD); while FIG. 2 is a diagrammatic view of medical device 10 as an implantable medical device (IMD) implanted in patient P. In both external and internal embodiments, medical device 10 includes controller 12 coupled to blood pressure sensor 14 via blood pressure medical lead 16. Controller 12 is further coupled to one or more electrodes 18 via electrode medical lead 20. Electrodes 18 are positioned to provide electrical stimulation of the cardiac afferent sympathetic nerves.

In operation, controller 12 monitors blood pressure via blood pressure sensor 14 and provides electrical stimulation to the cardiac afferent sympathetic nerves via electrodes 18 as a function of the sensed blood pressures. Controller 12 monitors blood pressures on a continuous, periodic, or non-periodic, on-demand basis to determine whether a current blood pressure is within normal levels. If blood pressures are normal, controller 12 need not take any action, but preferably continues monitoring blood pressures. If, however, blood pressures are outside normal levels, controller 12 selectively provides signals to electrodes 18 to cause blood pressures to return to normal levels. Specifically, if the sensed blood pressure falls below normal levels, controller 12 provides signals to electrodes 18 to block the cardiac afferent sympathetic nerves and, correspondingly, to increase blood pressure. Conversely, if controller 12 determines that blood pressure has risen above normal pressures, controller 12 provides signals to the cardiac afferent sympathetic nerves via electrodes 18 to stimulate the cardiac afferent sympathetic nerves and, corresponding, to decrease blood pressure.

Controller 12 may take the form of an external device or an implantable device. Controller 12 in the form of an external device may be particularly beneficial for patients acutely experiencing abnormal blood pressures (for example, in patients experiencing HELLP (Hemolysis, Elevated Liver, Low Platelet) Syndrome). For patients with more chronic blood pressure problems, however, controller 12 preferably is constructed in a housing intended for implant within the human body.

As shown in FIGS. 1 and 2, controller 12 is a single device. However, the present invention contemplates numerous configurations in which the functionality of controller 12 is divided between several distinct devices communicatively coupled to one another. For example, medical device 10 may comprise a blood pressure monitor that communicates with a separate electrical neurostimulator through telemetry or some other communication method. Additionally, the blood pressure monitor may include additional features, including pacing and/or defibrillation capabilities.

Blood pressure sensor 14 may be implemented with any external or implantable sensor or monitoring device that measures arterial blood pressure, either directly or indirectly. Because blood pressure sensors are very well known in the field of medical devices, these sensors are not described in detail herein. However, a discussion of several different types of pressure sensors can be found in U.S. Pat. Nos. 5,353,800 and 6,155,267, both assigned to Medtronic, Inc.

As shown in FIG. 1, blood pressure sensor 14 may be a non-invasive blood pressure monitor incorporating oscillometric measurements. Such monitors generally rely upon an inflatable cuff similar to a conventional sphygmomanometer placed about the upper arm. Other non-invasive blood pressure monitors include pulse oximeters. As shown in FIG. 2, blood pressure sensor 14 may be an implantable electronic pressure sensor for positioning in the heart (or a blood vessel) such as that used with the Medtronic CHRONICLE™ Implantable Hemodynamic Monitor (IHM). Common electronic pressure sensors include piezoelectric (or piezoresistive) pressure transducers and sensors with a capacitor that changes capacitance with pressure changes, such as is disclosed in U.S. Pat. No. 5,564,434 assigned to Medtronic, Inc.

As shown in the illustrated embodiments, blood pressure lead 16 electrically couples controller 12 to blood pressure sensor 14. In alternate embodiments, a telemetry circuitry may be employed to enable controller 12 to communicate with blood pressure sensor 14.

Also well known are electrodes 18 for use in providing nerve stimulation. As shown in FIG. 1, electrodes 18 may be conventional, surface-mounted electrodes positioned in proximity to the cardiac afferent sympathetic nerves, which are located between the second and third intercostal spaces. In one embodiment, electrodes 18 may take the form of those electrodes commonly used in conjunction with Transcutaneous Electrical Nerve Stimulation (TENS) units. These surface mounted electrodes may be fixed to the patient via any of a variety of conventional mechanical or adhesive mechanisms.

As shown in FIG. 2, electrodes 18 may be implanted within the body near or in contact with a left ventral ansa of the cardiac afferent sympathetic nerves. These nerves can be reached by opening the chest through the left second or third intercostal space. Electrodes 18 receive electrical signals from controller 12, which signals are then transmitted to the cardiac afferent sympathetic nerves to either stimulate or block activity therein. Electrodes 18 may be used with a neurostimulator, such as the Medtronic Itrel™ or the Medtronic Synergy™ devices in communication with a blood pressure monitoring device.

As described above, the cardiac afferent sympathetic nerves are stimulated or blocked depending upon whether the blood pressure is below or above, respectively, normal blood pressures. Accordingly, controller 12 uses information received from blood pressure sensor 14 to control certain parameters of the signals transmitted to electrodes 18. These parameters include the amplitude, duration, duty cycle, frequency, and waveform shape of the signal. Typically, the stimulation will fall in the range of about 40-400 microsecond duration pulses, at a frequency in the range of about 10-100 Hz, and at a voltage of about 1-10 V. To block, or cause withdrawal of, cardiac sympathetic activity, controller 12 may select stimulation parameters (e.g., amplitude and waveform shape) from within a window of values known to obtain blocking. The value of these stimulation parameters may also vary depending upon the severity of the blood pressure variation. Stimulation of the cardiac afferent nerves can be performed using either external (FIG. 1) or implanted (FIG. 2) electrodes; however, current technology allows for blocking of cardiac afferent nerve activity only through implanted electrodes.

It should be appreciated that, owing to physiological differences between patients, an adjustment to the stimulation/blocking signal parameters may not produce an immediate, precise change in all patients. Rather, it is anticipated that each patient will respond substantially uniquely to variations in the stimulation/blocking signal parameters. Thus, it may be useful to add controllable variability to the operation of the feedback arrangement described herein. For example, it may be useful to control the rate at which the stimulation/blocking signal parameters are allowed to change, or to develop a histogram for a particular patient. The inventive system can include the ability to record parameters associated with the delivered stimulation/blocking signal such as amplitude, duration, duty cycle, frequency, and/or waveform shape. These parameters and the patient's response may be recorded in memory 36, for example. Based on patient response, the efficacy of the stimulating/blocking signal can be evaluated so that subsequently delivered stimulating/blocking signal can be adjusted to better control blood pressure. This “learning” capability allows the system to optimize stimulation/blocking signal parameters based on prior patient data so that treatment is automatically tailored to individual patient needs.

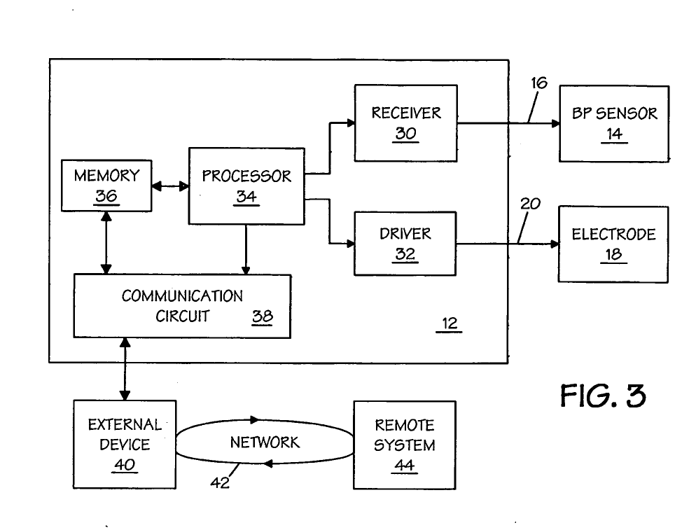

FIG. 3 is a functional block diagram of one embodiment of controller 12. This block diagram is intended to be merely exemplary and corresponds only to a general functional organization of a controller for use with the present invention. Controller 12 generally includes receiver circuit 30, driver circuit 32, processor 34, and memory 36.

Receiver circuit 30 is generally responsible for receiving signals from blood pressure sensor 14 via blood pressure lead 16, and for processing those signals into a form, such as a digital format, that may be analyzed by processor 34 and/or stored in memory 36. Driver circuit 32 is generally responsible for providing signals as directed by processor 34 to electrodes 18 via electrode leads 20. Processor 34, operating under software and/or hardware control, processes blood pressure signals received by receiver circuit 30 to determine whether current blood pressure is within normal blood pressures. Based upon the current blood pressure, processor 34 may instruct driver circuit 32 to produce an electrical signal having a specific set of parameters, such as amplitude, duration, duty cycle, frequency, and waveform shape, to either stimulate or block activity in the cardiac afferent sympathetic nerves to affect the blood pressure of patient P. Memory 36, in addition to storing blood pressure data, may store software used to control the operation of processor 34.

In one embodiment of controller 12, signals stored in memory 36 may be transferred via communication circuit 38, such as a telemetry circuit, to external device 40, such as a programmer. These signals may be stored in the external device, or transferred via network 42 to a remote system 44 which may be a repository or some other remote database. Network 42 may be an intranet, the Internet, or any other type of communication link.

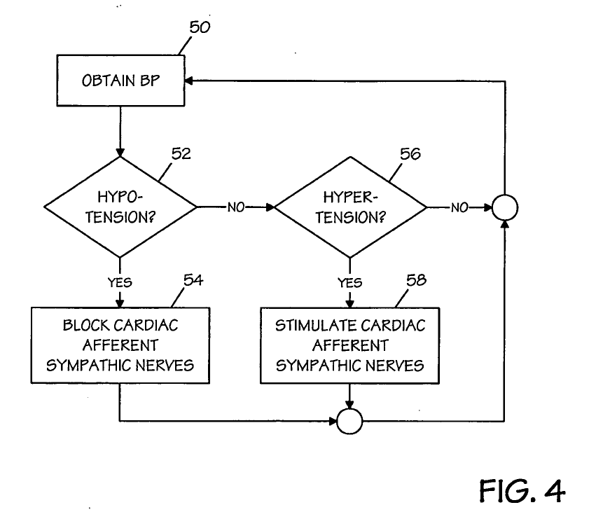

The overall general operation of processor 34 may be appreciated by reference to a flowchart depicted in FIG. 4. Those skilled in the art will appreciate that the flowchart illustrated herein may be used to represent either software that may be executed by processor 34 or hardware configured to perform the functions set forth in the flowchart.

The process illustrated in FIG. 4 begins at step 50 with processor 34 obtaining the current blood pressure value from receiver 30. At step 52, processor 34 evaluates the current blood pressure to determine if the patient is suffering from hypotension, or low blood pressure. Generally, for a given patient P, a range of normal, healthy blood pressures is stored in memory 36. Processor 34 then compares the current blood pressure to the patient's normal range of blood pressures to determine whether the current blood pressure has fallen below of the patient's normal range.

If it is determined at step 52 that patient P is hypotensive, processor 34 at step 54 instructs driver circuit 32 to produce an electrical signal having certain parameters for transmission to electrode 18 for blocking activity in the cardiac afferent sympathetic nerves of patient P. Blocking of the cardiac afferent nerve stimulates the arterial baroreceptor reflex system control of renal sympathetic activity. Increased renal sympathetic nerve activity decreases the renal excretory function. The effects of increased renal sympathetic nerve activity include increased renal tubular sodium reabsorption and renal sodium retention, decreased renal blood flow and glomerular filtration rate, increased renal vascular resistance, and increased renin release. As a result, the kidneys excrete less liquid, and the patient's blood pressure rises. After blocking step 54, the process returns to step 50 to begin the process again with the current blood pressure again being obtained. Here, if patient P is still determined at step 52 to be hypotensive, processor 34 will again instruct driver circuit 32 to deliver a signal for blocking nervous activity in the cardiac sympathetic nerves. This process is repeated until the patient's blood pressure returns to normal.

If it is determined at step 52 that patient P is not suffering from low blood pressure, then at step 56, processor 34 determines whether patient P is suffering from hypertension, or high blood pressure. Here, processor 34 compares the current blood pressure to the patient's normal range of blood pressures to determine whether the current blood pressure has risen above of the patient's normal range.

If it is determined at step 56 that patient P is suffering from high blood pressure, then at step 58, processor 34 instructs driver circuit 32 to produce an electrical signal having certain parameters for transmission to electrode 18 for stimulating activity in the cardiac afferent sympathetic nerves of patient P. As expected, the result of stimulating nervous activity is opposite of the result obtained by blocking nervous activity. Stimulating nervous activity in the cardiac afferent sympathetic nerves results in decreased renal sympathetic nerve activity, which in turn results in the kidneys excreting additional liquid, thereby causing the patient's blood pressure to decrease. After delivery of the blocking electrical signal, the process returns to step 50 to begin the process again with the current blood pressure again being obtained. Here, if patient P is still determined at step 56 to be hypertensive, processor 34 will again instruct driver circuit 32 to deliver a signal for stimulating nervous activity in the cardiac sympathetic nerves. This process is repeated until the patient's blood pressure returns to normal.

In practice, the present invention may be used to treat problems caused by both high and low blood pressure. For example, severe heart failure is associated with cardiac afferent sympathetic nerve stimulation. This activation of the cardiac sympathetic afferent nerves contributes to hypertension and arrhythmogenesis by reducing baroreceptor reflex system control of kidney function regulating pressure and electrolyte balance, which in turn leads to progression of heart failure. By blocking signals to the baroreceptor reflex system, the invention will allow the kidneys to function in a more normal manner.

Patients suffering from hypertension often use anti-hypertensive drugs to decrease blood pressure. However, about 10-20% hypertension patients are drug-refractory, meaning the drugs are not effective in treating them. Also, anti-hypertensive drugs may have undesirable side effects. The present invention may be used to help patients who are drug-refractory to maintain normal blood pressure.

At the other end of the blood pressure spectrum, orthostatic hypotension is low blood pressure that causes dizziness, faintness or lightheadedness that appears only upon standing. This is caused by improper functioning of the baroreceptor reflex system. To treat orthostatic hypotension, both baroreceptor reflex system regulation of the kidneys and baroreceptor reflex system regulation of the heart should be affected. Blocking of afferent nerves from the heart influence the baroreceptor reflex system, which in turn activates efferent nerves to the heart. Efferent nerves to the heart affect heart rate, force of contraction and duration of contraction of the heart. The invention thus affects both the heart and the kidneys to help patients who suffer from orthostatic hypotension to maintain a normal blood pressure.

Kidney disease is associated with hypertension. Primary kidney damage leads to an increase in blood pressure, which in turn further induces an increase in blood pressure by damaging the remaining viable part of the kidney until end-stage renal disease develops. To prevent hypertension, kidney patients are often placed on anti-hypertensive drugs. However, kidney dialysis results in decreased blood pressure. Thus, if kidney patients are on anti-hypertensive drugs, they may suffer from temporary hypotension. Hypotension compromises the functioning of the already failing kidneys. The fact that dialysis patients are confronted with periods of both hypotension and hypertension increases the need to regulate blood changes, as well as kidney function, in these patients. Furthermore, hypertension in patients with kidney failure should be prevented in order to prevent further deterioration of the kidneys. The present invention can be used by kidney disease patients both to maintain normal blood pressure and to maintain normal functioning of the kidneys.

Patients suffering from heart problems and patients undergoing hemodialysis both suffer from electrolyte imbalances caused by improper functioning of the kidneys. The present invention can be used to normalize the electrolyte balance in these patients by stimulating the cardiac afferent nerves innervating the baroreceptor reflex system.

The present invention normalizes blood pressure by stimulating and/or blocking nervous activity in the cardiac afferent sympathetic nerves. Blood pressure is monitored (on a continuous, periodic, or on-demand basis) and, if it falls below a normal range of blood pressures, a left ventral ansa of the cardiac afferent sympathetic nerves is stimulated. This results in activation of sympathetic nerves to the kidney, which results in increasing blood pressure. If blood pressure is too high, activity in the cardiac afferent sympathetic nerves is blocked, resulting in a reduction in blood pressure.

Although the present invention has been described with reference to preferred embodiments, workers skilled in the art will recognize that changes may be made in form and detail without departing from the spirit and scope of the invention.

1. A medical device, comprising:
a blood pressure sensor for sensing current blood pressure;
an electrode for placement in proximity to cardiac afferent sympathetic nerves; and
a controller for providing an electrical signal to the electrode as a function of the sensed blood pressure.

2. A medical device according to claim 1, wherein at least one parameter defining the electrical signal is selected by the controller to cause electrical activity in the cardiac afferent sympathetic nerves to be stimulated.

3. A medical device according to claim 2, wherein the at least one parameter is selected from at least one of: an amplitude metric, a pulse width duration metric, a duty cycle, a frequency, a waveform shape.

4. A medical device according to claim 1, wherein at least one parameter defining the electrical signal is selected by the controller to cause electrical activity in the cardiac afferent sympathetic nerves to be blocked.

5. A medical device according to claim 1, wherein the blood pressure sensor comprises an implantable blood pressure sensor.

6. A medical device according to claim 5, wherein the implantable blood pressure sensor comprises a pressure transducer for placement in the heart or a blood vessel.

7. A medical device according to claim 1, wherein blood pressure sensor comprises an external blood pressure sensor.

8. A medical device according to claim 1, wherein the electrode comprises an implantable electrode.

9. A medical device according to claim 1, wherein the electrode comprises an external electrode.

10. A method for affecting blood pressure, the method comprising:
sensing a current blood pressure;
electrically blocking electrical activity in the cardiac afferent sympathetic nerves if the sensed blood pressure is below a range of normal blood pressures; and
electrically stimulating electrical activity in the cardiac afferent sympathetic nerves if the blood pressure is above the range of normal blood pressures.

11. A method according to claim 10, wherein the method is prescribed as therapy for a hemodialysis patient.

12. A method according to claim 10, wherein the method is prescribed as therapy for an orthostatic hypotension patient.

13. A method according to claim 10, wherein the method is prescribed as therapy for a heart failure patient.

14. A method according to claim 10, wherein the method is prescribed as therapy for a patient suffering from one of drug-refractory hypertension and drug-refractory hypotension.

15. A method according to claim 10, wherein the method is performed by an implantable medical device.

16. A medical device, comprising:
a pressure sensor configured to sense blood pressure;
a receiver circuit for receiving from the pressure sensor a blood pressure signal indicative of sensed blood pressure;
an electrode placed in proximity to cardiac afferent sympathetic nerves;
a driver circuit for providing a stimulating/blocking signal to the electrode; and
a processor for controlling at least one parameter of the stimulating/blocking signal as a function of sensed blood pressure.

17. A medical device according to claim 16, wherein the receiver circuit processes and digitizes the blood pressure signal.

18. A medical device according to claim 17, and further comprising memory for storage of the digitized blood pressure signal.

19. A medical device according to claim 16, and further comprising a communication circuit for communication with device separate from the medical device.

20. A medical device according to claim 16, wherein the electrode is placed in proximity with a left ventral ansa of the cardiac afferent sympathetic nerves.
